(19) United States
(12) Patent Application Publication
(10) Pub. No.: US 2013/0157308 A1
(43) Pub. Date: Jun. 20, 2013

(54) BIDIRECTIONAL PROMOTER REPORTER VECTOR FOR THE ANALYSIS OF DUAL REGULATORY ELEMENTS

(71) Applicant: UNIVERSITY OF SOUTH CAROLINA, Columbia, SC (US)

(72) Inventors: David Reisman, Columbia, SC (US); Amanda Polson, Lexington, SC (US)

(73) Assignee: UNIVERSITY OF SOUTH CAROLINA, Columbia, SC (US)

(21) Appl. No.: 13/713,622

(22) Filed: Dec. 13, 2012

Related U.S. Application Data
(60) Provisional application No. 61/630,603, filed on Dec. 15, 2011.

(51) Int. Cl. C12N 15/85

(52) U.S. Cl. CPC C12N 15/85
USPC 435/69.1; 435/320.1; 435/325; 435/366; 435/354

(57) ABSTRACT

A bidirectional expression vector is described that can be utilized to determine the existence and characteristics of bidirectional promoters. The bidirectional expression vector includes two different reporter genes in a head to head (5′ to 5′) arrangement. In addition, the bidirectional expression vector can include a polylinker region located between the heads of the two reporter genes that provides multiple cloning sites for nonexclusive examination of polynucleotide sequences. The vector can also include a splicing site and drug resistance. The bidirectional expression vector can be used to examine a polynucleotide sequence for the presence of divergent regulator regions and, following determination of a bidirectional promoter, can be utilized to further elucidate characteristics of the bidirectional promoter.

### CROSS REFERENCE TO RELATED APPLICATION

This application claims filing benefit of U.S. Provisional Patent Application Ser. No. 61/630,603 having a filing date of Dec. 15, 2011, the contents of which is incorporated herein for all purposes.

### GOVERNMENT SUPPORT CLAUSE

This invention was made with government support under 2P20RR016461-10 awarded by National Institutes of Health. The government has certain rights in the invention.

### BACKGROUND

The process of transcribing DNA to RNA is accomplished by the recognition of specific elements on the DNA by general transcription factors that define the proper location(s) for RNA polymerase II (RNAPII) to initiate transcription (Buratowski, 2008). The binding of transcription factors to the promoter region of genes stimulates the modification and rearrangement of the chromatin structure and forms the pre-initiation complex (Seila, et al, 2009). Many recent experimental results have provided evidence that transcription can occur in a divergent manner, meaning transcription initiation events can occur in opposite directions from a single promoter region (Seila, et al, 2009).

Previous computational analysis of many vertebrate genomes has identified a class of regulatory regions that contain an arrangement referred to as being in a head-to-head (5′ to 5′) orientation on opposite strands of the DNA (Wang, et al, 2009; Lapidot and Pipel, 2006; Trinklein, et al, 2004). These regulatory regions often have fewer than 1000 base pairs separating their corresponding transcription start sites and have been termed as being “bidirectional” (Wang, et al, 2009; Yang, et al, 2008; Yang and Elnitski, 2008; Trinklein, et al, 2004). This bidirectional arrangement and the divergent gene pairs (bidirectional genes) under the control of these regulatory regions (bidirectional promoters), are a common feature within not only the human genome but also in other genomes such as mouse, yeast, and plant genomes (Seila, et al, 2009; Wang, et al, 2009).

Since divergent transcription occurs more often throughout the genome than was previously thought, defining features within the genome that could serve as divergent or bidirectional promoters could be of importance in functionally understanding how these particular elements operate. Shared bidirectional regulatory regions, such as gene promoters, can influence the expression of the oppositely oriented genes, yet many of the specifics as to how this process occurs is unknown (Pointkivska, et al, 2009). Understanding the significance of the function(s) of these bidirectional promoters is important to identifying the way bidirectional genes are regulated and expressed (Wang, et al, 2009).

While some bidirectional expression vectors have been reported previously (Wright, et al, 1995; Ueda, et al, 2006; Zanotto, et al, 2006), problems exist with these vectors. For instance, the vector components utilized can lead to the necessity of complicated assay techniques that can often present ambiguous results.

Accordingly, what is needed in the art is a construct that can be utilized to examine genetic elements to determine the presence and activity of bidirectional promoters within the genome.

### SUMMARY

According to one embodiment, disclosed is a bidirectional expression vector. The bidirectional expression vector includes a first reporter gene and a second reporter gene, the first and second reporter genes being arranged in a head to head (5′ to 5′) arrangement. The bidirectional expression vector also includes a polylinker region between the heads of the first and second reporter genes, and includes multiple cloning sites within the polylinker region.

Also disclosed is a method for the production of a first expression product of a first reporter gene and a second expression product of a second reporter gene from a bidirectional vector, the method including a polynucleotide into a cloning site of the bidirectional vector, transfecting a host cell with the bidirectional vector, and culturing the host cell.

### BRIEF DESCRIPTION OF THE FIGURES

A full and enabling disclosure of the present invention, including the best mode thereof to one skilled in the art, is set forth more particularly in the remainder of the specification, which includes reference to the accompanying figures, in which:

FIG. 1 illustrates the structure of the bidirectional expression vector described herein.

FIG. 2 is a representation of the chromosomal location of the human p53 and WDR79 genes as identified through GenBank.

FIG. 3 includes maps showing the structures of the human (FIG. 3A) and mouse (FIG. 3B) p53 promoters and the known transcription factor binding sites along the two promoters.

FIG. 4 graphically illustrates individual p53 and WDR79 promoter activities including human (FIG. 4A) and mouse (FIG. 4B) p53 and WDR79 promoters following cloning into a pGL3 vector.

FIG. 5 illustrates the p53 and WDR79 promoter activities in the bidirectional expression vector for mouse (FIG. 5A) and human (FIG. 5B) bidirectional p53 and WDR79 promoters following cloning of the promoters into the bidirectional expression vector.

FIG. 6 illustrates the results following cloning of the unidirectional human Bax promoter into the bidirectional expression vector in the Bax to Ren orientation (FIG. 6A) and the Bax to Luc orientation (FIG. 6B).

FIG. 7 illustrates the results following cloning of mouse or human p53 and WDR79 bidirectional promoters into the bidirectional expression vector and transient transfections performed in murine Swiss3T3 (FIG. 7A) and LTK (FIG. 7B) cell lines and human HeLa (FIG. 7C), HT29 (FIG. 7D), and T98G (FIG. 7E) cells lines.

FIG. 8 illustrates the results following RNA purification, reverse transcription and PCR amplification of Endogenous p53 and WDR79 Levels in Murine Swiss 3T3 Cells Upon Entry into S-Phase with specified primers corresponding to the murine p53 promoter (FIG. 8A) or the murine WDR79 promoter (FIG. 8B), with PCR amplification of β-actin as a control (FIG. 8C).

### DETAILED DESCRIPTION

The following description and other modifications and variations to the present invention may be practiced by those of ordinary skill in the art, without departing from the spirit and scope of the present invention. In addition, it should be understood that aspects of the various embodiments may be interchanged in whole or in part. Furthermore, those of ordinary skill in the art will appreciate that the following description is by way of example only, and is not intended to limit the invention.

The present disclosure is generally directed to a bidirectional expression vector that can be beneficially utilized to examine genetic regulatory regions for the presence of divergent or bidirectional promoters as well as to better understand the structure and function of bidirectional promoters. For example, the bidirectional expression vector can beneficially be utilized to study the transcriptional activities of potential bidirectional promoters. In addition, the bidirectional expression vector can be utilized to discover factors involved in the regulation of the bidirectional genes under the control of the divergent promoters. Moreover, the bidirectional expression vector can be used to study the transcriptional output of each bidirectional promoter individually. This can allow researchers to determine the output of transcripts mediated by the bidirectional promoters.

The wide ranging capabilities of the bidirectional expression vector can be utilized in determining the characteristics of bidirectional promoters following initial identification of the promoters. For example, following recognition of a bidirectional gene pair, mutations can be incorporated within the regulatory regions and the bidirectional expression vector can be used to study the effect(s) of these mutations on the promoters, as well as examining the binding of cell-specific factors involved in their regulation. For example, deletions and point mutations can be created within the promoters following initial recognition. The activity of the promoters with the deleted sequences can then be examined to assess whether or not they are capable of driving expression of the genes in the absence of those deleted sequences and to assess whether there are important regulatory regions within the promoters that are essential for the expression of the bidirectional transcripts.

The bidirectional expression vector is a nonexclusive vector that can incorporate any polynucleotide for examination. The bidirectional expression vector offers several advantages to researchers, such as multiple cloning sites within a poly-linker region, the presence of a splicing site, multiple restriction sites, drug resistance, and the incorporation of two different reporter genes, the expression products of which can be assayed according to any of a variety of straight forward and well-known methods. Thus, the vector can be utilized to examine essentially any genetic sequence for the presence of bidirectional promoters. Moreover, once the bidirectional promoters are known, the transcriptional activity of each individual promoter can be measured in a relatively straight-forward fashion through utilization of the vector.

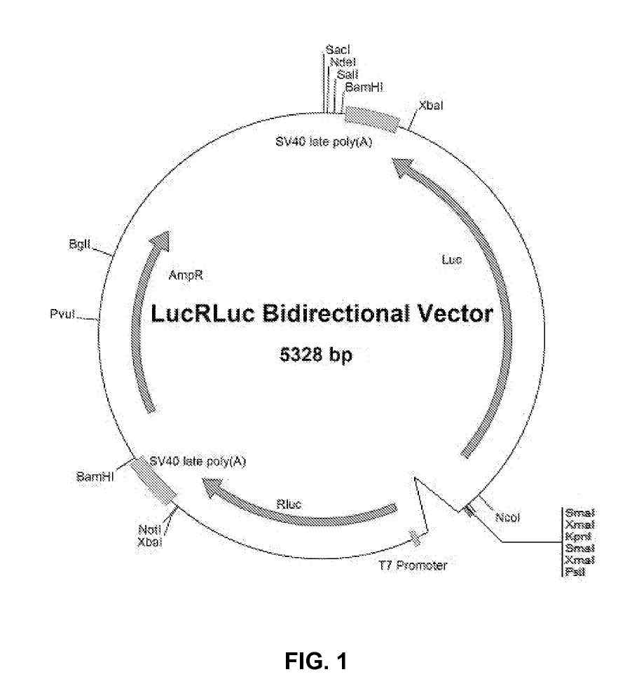

Referring to FIG. 1 one embodiment of the bidirectional expression vector is illustrated. As can be seen the bidirectional expression vector includes two reporter genes in a head to head (5′ to 5′) arrangement with a polylinker region, as shown, between the heads of the two reporter genes. In this particular embodiment, the reporter genes are the widely used renilla (Lorenz, et al, 1991) and luciferase reporter genes (De Wet, et al, 1986). It should be understood, however, that while the specific embodiment shown in FIG. 1 incorporates the luciferase and renilla reporter genes, the bidirectional expression vector is not limited to these particular reporter genes, and other reporter genes may alternatively be utilized in the vector, provided that the different reporter genes are provided in a head to head arrangement.

As utilized herein, the term “reporter gene” generally refers to a gene the expression product of which can be detected quantitatively, for instance through emission of a signal (fluorescence, etc.) for which the intensity differs according to the amount of gene expression. Reporter genes as may be incorporated in the bidirectional expression vector can include, for example, a renilla gene, a luciferase gene, a CAT gene, a lacZ gene and so forth such as a firefly luciferase gene and a renilla luciferase gene.

In addition to the two reporter genes arranged in a head to head arrangement, the bidirectional expression vector includes a polylinker region between the heads of the two reporter genes that can provide multiple cloning sites for insertion of a polynucleotide that is to be examined for the presence and/or activity of a bidirectional promoter region. The multiple cloning sites and the fact that the vector has no proper reading frame requirement can provide the bidirectional expression vector with the flexibility to incorporate essentially any nucleotide sequence for examination. This broad application capability and ease of use in examining polynucleotide regions of interest for the presence and/or activity of bidirectional promoters can be of great benefit to researchers.

The polylinker region can include a plurality of restriction sites for cloning. By way of example, restriction sites as may be included in the polylinker region can include, without limitation, two or more of KpnI, SmaI, XmaI, PstI, SalI, NdeI, SacI, XhoI, and/or BgII. In addition, the polylinker region can include more than one of each restriction site. For example, the polylinker region can include the following restriction sites in this order: SmaI, XmaI, KpnI, SmaI, XmaI, and PstI, as illustrated in FIG. 1. In another embodiment, the polylinker region can include at least one copy of each of SmaI, KpnI, SacI, SalI, XhoI, and BgII. The polylinker region can generally include about 5 restriction sites or more, for instance, between about 5 and about 15 restriction sites for cloning.

The bidirectional expression vector can also include a prokaryotic antibiotic resistance gene such as the ampicillin resistance gene or the kanamycin resistance gene. As shown in FIG. 1, the antibiotic resistance gene (in this case the ampicillin resistance gene AmpR) can be downstream of one of the reporter genes. In this embodiment, the ampicillin resistance gene is downstream of the renilla reporting gene, though this is not a requirement of the vector, and an antibiotic resistance gene can alternatively be located at any suitable site on the vector.

The vector may also include one or more further elements, such as enhancer elements, splicing signals, polyadenylation signals, termination signals, RNA export elements, secretion signals, internal ribosome entry sites, and so forth. For example, the expression vector may comprise a polyadenylation (poly(A)) signal for stabilization and processing of the 3′ end of the reporter gene transcript. Poly(A) signals include, for example, the rabbit beta globin poly(A) signal or the bovine growth hormone poly(A) signal, as well as poly(A) signals of viral origin, such as the SV40 late poly(A) region. In one embodiment, the vector comprises a chicken beta globin terminator/poly(A) signal. A poly(A) signal may be included downstream of each reporter gene, as shown in the vector of FIG. 1. In some embodiments, restriction sites may also be included downstream of the poly(A) signal.

The disclosure also encompasses vectors wherein any or all of the elements as described herein are replaced by functional variants of said elements as are known in the art.

A further aspect of the disclosure relates to a host cell or host cell line comprising the bidirectional expression vector. As utilized herein, the term “host cell” generally refers to any cell that may be transfected with the bidirectional expression vector. For example, a host cell may be a mammalian cell, a bacterial cell, a yeast cell or an insect cell.

In one embodiment, the host cell can be a mammalian cell. Examples of mammalian host cells include, by way of nonlimiting example, Chinese hamster ovary (CHO) cells, CHO-K1 cells, CHO-DXB-11 cells, CHO-DG44 cells, bovine mammary epithelial cells, mouse Sertoli cells, canine kidney cells, buffalo rat liver cells, human lung cells, murine mammary tumor cells, murine Swiss3T3 cells, murine LTK cells, murine 10(3) cells, rat fibroblasts, bovine kidney (MDBK) cells, NSO cells, SP2 cells, TR1 cells, MRC 5 cells, FS4 cells, HEK-293T cells, NIH-3T3 cells, HeLa cells, HT-29 cells, T98G cells, U2OS cells, baby hamster kidney (BHK) cells, African green monkey kidney (COS) cells, human hepatocellular carcinoma (e.g., Hep G2) cells, A549 cells, etc. Mammalian host cells can be cultured according to methods known in the art (see, e.g., J. Immunol. Methods 56:221 (1983), Animal Cell Culture: A Practical Approach 2nd Ed Rickwood, D. and Hames, B. D., eds. Oxford University Press, New York (1992)).

The bidirectional expression vector can also be introduced into a bacterial cell. In one embodiment, competent E. coli can be transformed. Examples of suitable E. coli include, without limitation, DH1, DHS, DH5α, XL1-Blue, SURE, SCS110, OneShot Top 10, and HB101.

The bidirectional expression vector may be introduced into a host cell according to any of the many techniques known in the art, e.g., dextran-mediated transfection, polybrene-mediated transfection, protoplast fusion, electroporation, calcium phosphate co-precipitation, lipofection, direct microinjection of the vector into nuclei, or any other means appropriate for a given host cell type.

A further aspect of the disclosure relates to a method for the production of the expression product of the reporter genes, comprising the steps of: a) cloning a polynucleotide region of interest into the bidirectional vector; b) transfecting a host cell or host cell line (e.g., a mammalian host cell or host cell line) with the bidirectional expression vector; b) culturing the cell under appropriate conditions to enable growth and/or propagation of the cell and expression of the reporters; and, optionally c) determining the presence or quantity of the expressed reporters.

Methods for determining the presence or quantity of the expressed reporter can depend upon the specific nature of the reporter. By way of nonlimiting example, RNA of the two reporters can be analyzed according to reverse transcription and PCR amplification, as is generally known. Alternatively, cellular extracts can be assayed to determine reporter activity. For example, protein expression products of the reporter genes can be isolated and/or purified from one another and other biological material by salt or alcohol precipitation (e.g., ammonium sulfate precipitation or ethanol precipitation), affinity chromatography (e.g., used in conjunction with a purification tag); fractionation on immunoaffinity or ion-exchange columns; high pressure liquid chromatography (HPLC); reversed-phase HPLC; chromatography on silica or on a cation-exchange resin such as DEAE; chromatofocusing); isoelectric focusing; countercurrent distribution; SDS-PAGE; gel filtration (using, e.g., Sephadex G-75); and protein A Sepharose columns to remove contaminants such as IgG. Such purification methods are well known in the art and are disclosed, e.g., in “Guide to Protein Purification”, Methods in Enzymology, Vol. 182, M. Deutscher, Ed., 1990, Academic Press, New York, N.Y. When cellular products are examined for the presence or quantity of the protein expression products of the reporter genes, it may be beneficial to include one or more inhibitors of proteolytic enzymes in an assay system, such as phenylmethanesulfonyl fluoride (PMSF), Pefabloc SC, pepstatin, leupeptin, chymostatin and EDTA.

Growth of mammalian cells in liquid aqueous culture is well known in the art. Examples of mammalian cell culture growth media which are known in the art include, without limitation, EX-CELL ACF, CHO medium (Sigma-Aldrich (St. Louis, Mo.), DMEM, DMEM/F-12, F-10 Nutrient Mixture, RPMI Medium 1640, F-12 Nutrient Mixture, Medium 199, Eagle's MEM, RPMI, 293 media, and Iscove's Media. Cell growth can be performed in any of several systems. For example, cell growth can be done in a simple flask, e.g., a glass shake flask. Other systems include tank bioreactors, bag bioreactors and disposable bioreactors.

The bidirectional expression vector may be provided in a kit. The kit may include, in addition to one or more vectors, any reagent which may be employed in the use of the vector. In one embodiment, the kit includes reagents necessary for cloning of a polynucleotide region of interest into the vector and transformation of the vector into cells. For example, the kit may include reagents for a calcium phosphate transformation procedure: calcium chloride, buffer (e.g., 2×HEPES buffered saline), and sterile, distilled water. In another embodiment, the kit includes reagents for a DEAE-Dextran transformation: Chloroquine in PBS, DEAE-dextran in PBS and Phosphate buffered saline. In yet another embodiment, reagents for a liposome transformation are included in the kit: Liposomes extruded from DOTAP/cholesterol extruded liposomes. For example, the kit may include the cationic lipid-based transfection reagent Lipofectamine™ (Invitrogen Life Technologies; Carlsbad, Calif.).

A kit may include reagents required for bacterial transformation of the bidirectional expression vector. For example, the kit may include transformation competent bacteria (e.g., DH1, DHS, DH5a, XL1-Blue, SURE, SCS110, OneShot Top 10, or HB101).

A kit may include growth media or reagents required for making growth media. For example, in one embodiment, a kit can include fetal calf serum or DMEM (Dulbecco/Vogt modified Eagle's (Harry Eagle) minimal essential medium) for growth of mammalian cells. In another embodiment, a kit can contain powdered Luria broth media or Luria broth plates containing an appropriate antibiotic (e.g., ampicillin or kanamycin) for growing bacteria. Components supplied in a kit may be provided in appropriate vials or containers (e.g., plastic or glass vials). The kit can include appropriate label directions for storage, and appropriate instructions for usage.

Since there is a significant amount of divergent transcription thought to be occurring within the genome, identifying strategies to study this phenomenon is of great importance. Development of the bidirectional expression vector is one such tool that can be incorporated to aid in the understanding and discovery of how these bidirectional regulatory regions are functioning to transcribe their corresponding genes. Understanding exactly how this process occurs could play a significant role in knowing how bidirectional gene partners may be coordinately regulated or possibly regulate each other's expression.

The bidirectional expression vector provides a way to measure gene activity of bidirectional regulatory regions, i.e. bidirectional promoters. This can prove to play a significant role in our understanding of how bidirectional promoters can potentially regulate the expression of their oppositely oriented gene partner. Since the roles bidirectional transcripts play in the regulation of gene expression are not sufficiently understood, the bidirectional expression vector can allow for the further understanding of how the processes of bidirectional transcription occurs.

The present disclosure may be better understood with reference to the Example, below.

### Example

The p53 protein is an important tumor suppressor that is associated with many cellular processes including, regulation of the cell cycle, DNA repair, transcriptional regulation of genes, chromosomal segregation, cell senescence, and apoptosis (Vogelstein et al., 2000; Zhao, et al., 2009). The p53 protein has the ability to maintain stability of the genome through the induction of several intracellular and extracellular factors (Vousden and Lu, 2002; Zhao, et al., 2009). When damage to DNA occurs, p53 expression goes up, due to increased stability of the p53 protein. The accumulation of p53 triggers a cascade of events that can lead to either cell cycle arrest or apoptosis of the cell (Vousden and Lu, 2002; Zhao, et al., 2009).

Transcriptional regulation of the p53 gene is important because its inactivation results in the loss of the apoptotic response and/or failure to undergo cell cycle arrest in response to cellular stress or DNA damage. It has been reported that, in many human cancers, p53 transcription is deregulated (Khoo, et al., 2009, Boggs and Reisman, 2007; Boggs and Reisman, 2006), whether it be increased (as is the case with mutant p53 transcription) (Balint and Reisman, 1996) or decreased (failure to activate transcription of wild type p53) (Raman, et al, 2000), and can contribute to the formation of human cancers (Vousden and Lu, 2002, Boggs and Reisman, 2006). Most of the information known about p53 is at the protein level. However, the molecular basis for transcriptional regulation of the p53 gene has been less well defined. Understanding the transcriptional regulation of the p53 gene can contribute to understanding of the mechanisms involved in regulating the overall levels of p53 protein.

Recently, a number of genes located near the p53 gene and arranged in the opposite orientation have been identified. The most well studied to date, termed Wrap53 partially overlaps the first exon of p53 and encodes an antisense transcript that regulates p53 post-transcriptionally (Mahmoudi, et al, 2009, Mahmoudi, et al, 2010). In addition, the Wrap53 gene undergoes complex alternative splicing, producing at least seventeen different splice variants (Mahmoudi, et al, 2009). One or more of the splice variants of Wrap53 encodes a WD40 domain protein that appears to be essential for Cajal body formation (Mahmoudi, et al, 2009; Mahmoudi, et al, 2010).

The origin of the transcripts encoding the Wrap53 protein appears to be complicated. Since the protein coding sequences initiate in exon 2 of the Wrap53 transcript, and are derived, in part, from transcripts that appear to initiate over 2000 base pairs (bp) downstream of the region of overlap with p53 exon 1, the protein may, in fact, be encoded by a separate transcription unit that has been identified previously as WDR79 (Mahmoudi, et al, 2009; Garcia-Closas, et al, 2007, Alonso, et al, 2009).

This example focuses on p53's closely separated bidirectional gene partner, WDR79, and the bidirectional vector described herein is utilized to determine whether the shared region between the TSS's of these two genes is a functional, bidirectional promoter. By focusing this example on the transcriptional regulation of p53 and its bidirectional gene partner, WDR79, key factors can be elucidated by use of the bidirectional expression vector that can control and regulate the expression of these genes.

### Bidirectional Expression Vector Preparation

The pRL-null vector (Promega) expressing the Renilla reporter gene (from the sea pansy Renilla reniformis, herein referred to as Renilla) (Lorenz, et al, 1991) was digested with EcoR1 restriction enzyme. The pGL3 vector (Promega), which expresses the Luciferase reporter gene (from the firefly Photinus pyralis, herein referred to as Luciferase) (De Wet, et al, 1986), underwent PCR amplification using primers specific for the Luciferase region of the vector (Luc Forward: 5′-GCTAGCCCGGGCTCGAGATCTGCG-3′ (SEQ ID NO.:1), Luc Reverse: 5′-GTCGACGGATCCTTATCGATTTTACCAC-3′ (SEQ ID NO.:2)). The amplified Luciferase fragment was cloned into the TOPO TA vector (Invitrogen) and digested with EcoRI. The Lucerifase fragment was gel purified and ligated to the EcoRI cut pRL-null vector. The newly synthesized vector expressing the two reporter genes in opposite orientations was from herein referred to as “pLucRLuc” bidirectional expression vector. The pLucRLuc vector was cut with SmaI. KpnI restriction linkers were cloned into the SmaI site of the vector so that the putatitive bidirectional promoters could be cloned at that position. (FIG. 1).

### PCR Amplification and Cloning of the WDR79 Promoter

The putative WDR79 promoter was PCR amplified from mouse and human genomic DNA using the following primers: Mouse WDR79 forward: 5′-GCCAGGTCAGGAGGGAGGCTATC-3′ (SEQ ID NO.:3), Mouse WDR79 Reverse: 5′-CATCTTCGTCGCGCTGAGTCCC-3′ (SEQ ID NO.:4); Human WDR79 Forward: 5′-GGAGTAGGCAGAAGACTCC-3′ (SEQ ID NO.:5), Human WDR79 Reverse: 5′-CAATCCAGGGAAGCGTGTCAC-3′ (SEQ ID NO.:6). The PCR fragments were cloned into the TOPO TA cloning vector (Invitrogen) and subcloned into the XhoI and KpnI sites of the pGL3 expression vector, transformed into JM109 cells (Promega), screened, and DNA was isolated from those samples that carried the WDR79 promoter region in the proper orientation. The p53 promoter was previously cloned in a similar manner using primers specific to the promoter region (Reisman, et al, 2001).

### Mouse and Human p53 Promoter and Human Bax Promoter Preparation

The 1.7 kbp mouse p53 promoter and the 1.1 kbp human p53 promoter were digested with XhoI, and treated with Klenow DNA polymerase. KpnI linkers were added to the excised promoter fragments. The bidirectional expression vector was digested with KpnI and the mouse or human p53/WDR79 promoters were ligated into the vector at the KpnI site.

As a control, the unidirectional human Bax promoter was digested out of the pGL3 vector with the restriction enzyme SmaI. The fragment was gel purified and ligated into the pLucRLuc bidirectional expression vector following digestion with the restriction enzyme SmaI.

### Cell Lines

Murine Swiss3T3, 10(3), and LTK, and human HeLa and T98G cells were grown in Dulbecco's Modified Eagle's Medium (DMEM) supplemented with 10% fetal bovine serum (FBS), 2 mM L-glutamine, 100 U/ml penicillin and 100 μg/ml streptomycin. Human HT29 and U2OS cells were grown in McCoy's 5A Medium supplemented with 10% fetal bovine serum (FBS), 2 mM L-glutamine, 100 U/ml penicillin and 100 μg/ml streptomycin. Cells were cultured at 37° C. in a humidified atmosphere of 6% CO₂ in air. The cells were grown until they were approximately 80% confluent. For induction of the cell cycle by serum treatment, Swiss3T3 cells were treated with DMEM containing 0.1% FBS for 24 hr. The cells were then stimulated to enter S-phase with DMEM containing 15% FBS and collected at the indicated time-points post-serum stimulation.

### RNA Extraction and RT-PCR Analysis

At indicated time points, serum-treated cultured cells were washed twice with PBS and harvested. RNA was extracted using the QIAGEN RNeasy protocol. RNA was reverse transcribed using Ambion RETROscript protocol and PCR amplifications were performed in a total volume of 50 μl. The samples were subjected to 30 amplification cycles. The sequences of the primers used are as follows (Invitrogen): p53 Forward: 5′-GGAAATTTGTATCCCGAGTATCTG-3′ (SEQ ID NO.:7), p53 Reverse: 5′-GTCTTCCAGTGTGATGATGGTAA-3′ (SEQ ID NO.:8), WDR79 Forward: 5′-GGATGCGGAGAGCCTATCGG-3′ (SEQ ID NO.:9), WDR79 Reverse: 5′-CATCCTGGTCGTCAACAGGG-3′ (SEQ ID NO.:10); and β-actin Forward/Reverse primer pair (Promega). The PCR products were verified by electrophoresis.

### Transient Transfection and Reporter Gene Assays

pGL3 expression vector: The murine 10(3) cell line was grown to confluency and split to 5×10⁴ cells/well in a 24 well plate. The cells were allowed to adhere overnight. The cells were transfected (TransFast Transfection Reagent; Promega) with various concentrations of the pGL3 vector containing the WDR79 and p53 promoter regions and harvested after an additional 24 hrs. Cellular extracts were obtained and Luciferase activity was assayed. All results were normalized to Renilla.

pLucRLuc Bidirectional Expression Vector:

Murine Swiss 3T3 and LTK, and human HeLa, T98G, and HT29 cell lines were grown to confluency and split to 5×10⁴ cells/well in a 24 well plate. The cells were allowed to adhere overnight. The cells were transfected with various concentrations (depending on the experiment being performed) of the bidirectional expression vector carrying either the unidirectional Bax promoter or the bidirectional p53/WDR79 gene promoters, a control pBSSK vector, and 1 μg of a β-Gal expressing vector (to use as an internal control; β-Gal expression is driven by the SV40 promoter). The cells were harvested 24 hrs later. Cellular extracts were obtained and Luciferase, Renilla, and β-Gal activity was assayed. All results were normalized to the β-Gal activity.

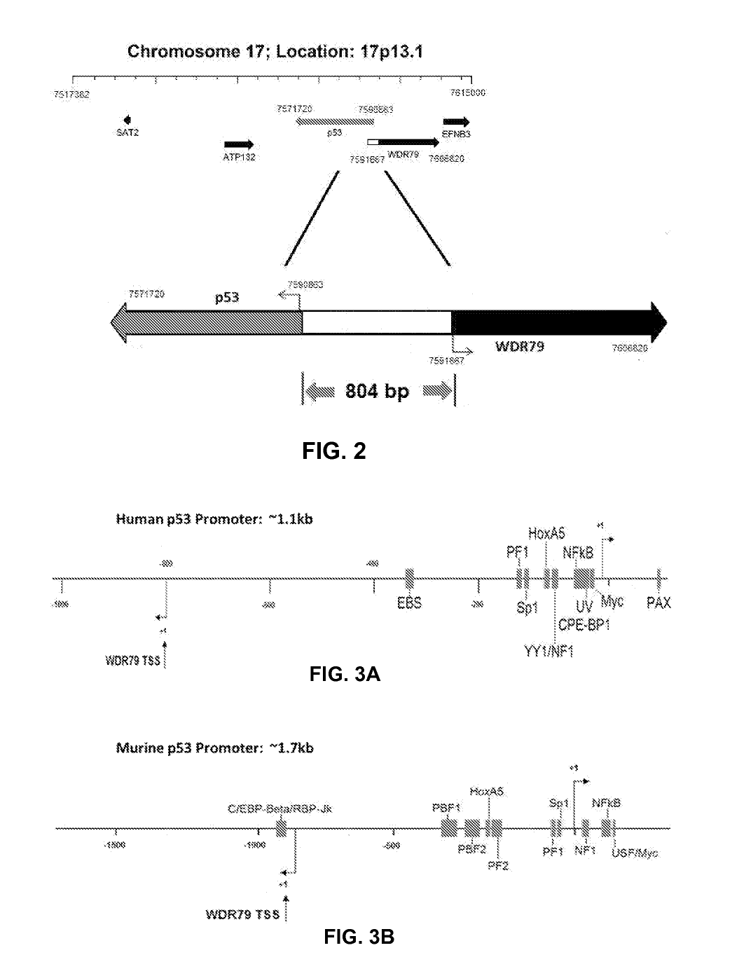

FIG. 2 illustrates the chromosomal location of the human p53 and WDR79 genes. As can be seen, p53 is flanked by the upstream gene, WDR79, in the opposite orientation. In FIG. 2, the numbers represent the nucleotide positions of the p53 and WDR79 genes on chromosome 17, and identify the transcription start site of each gene. The open white box on the WDR79 gene (arrow) is indicative of the overlapping region containing exon 1γ and 1α of the Wrap53 gene with the first exon of p53 in an antisense fashion as described by Mahmoudi, et al, 2009. The expanded region is representative of the p53 and WDR79 genes and the 804 bp regulatory region separating the two genes.

Because the promoter regions of both p53 and WDR79 are so closely located within the genome, it was first determined whether the two were located within the same region of DNA. The region was PCR amplified from genomic DNA, cloned into the TOPO TA cloning vector, and subcloned into the pGL3 expression vector. The expression vector containing either the putative human or mouse WDR79 promoter was transfected into murine 10(3) cells for 24 hrs. Transient transfections were performed in murine 10(3) cells. Titrations of increasing increments of the pGL3 vector containing the mouse and human p53 or WDR79 promoters were transfected. Each titration was performed in duplicate. The cells were harvested and cell extracts were obtained. The transcriptional activity of the p53 and WDR79 promoters were measured by transient transfections and reporter assays.

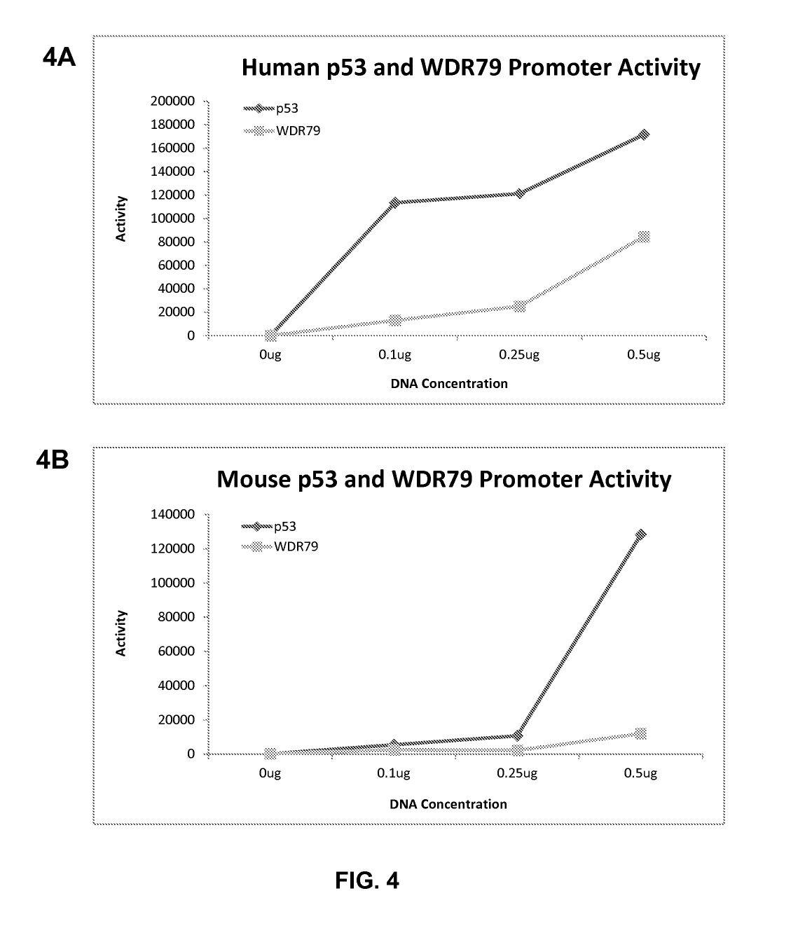

The activities of the human and mouse WDR79 promoter were compared to the activities of the human and mouse p53 promoters (FIG. 3A, 3B, respectively) which had previously been constructed in the manner as described above (Reisman, et al, 2001). FIG. 3 includes maps showing the structures of the human (FIG. 3A) and mouse (FIG. 3B) p53 promoters and the known transcription factor binding sites along the two promoters. The numbers shown on the map represent nucleotide positions and the transcription start site of the p53 gene.

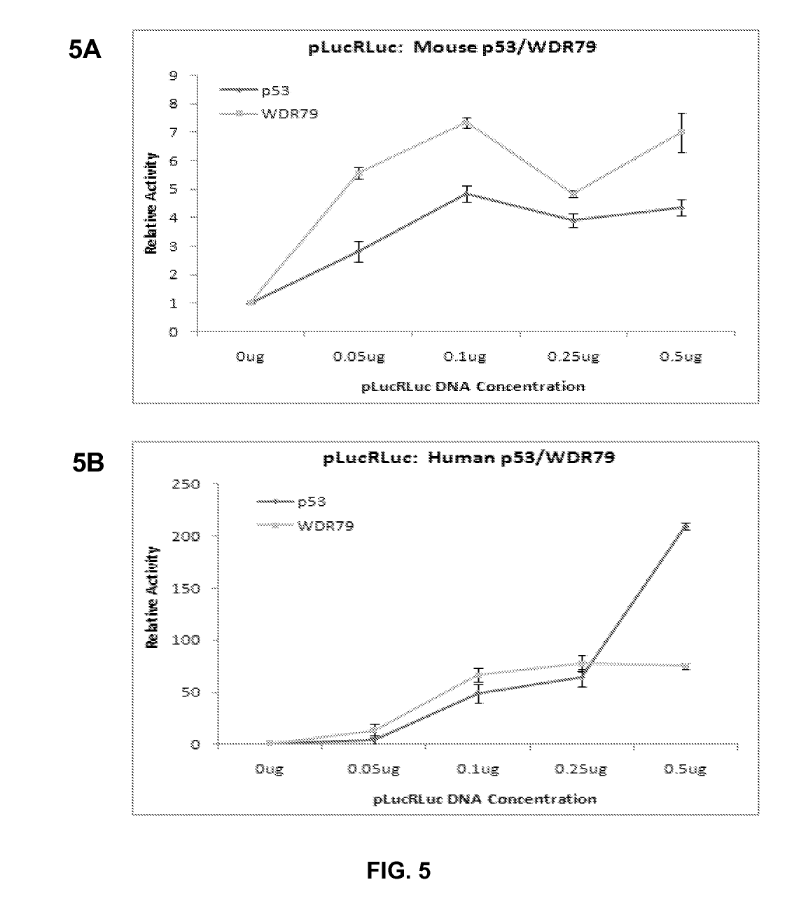

The p53 and WDR79 promoter activity was measured and the results were averaged. All results were normalized to Renilla. The results are shown in FIG. 4 for human (FIG. 4A) and mouse (FIG. 4B) p53 and WDR79 promoter activity in the pGL3 expression vector. As shown in FIG. 4A, the activities of the individual human WDR79 and p53 promoters show increasing activities in a dose dependent manner with the p53 promoter yielding higher activity than WDR79. Similarly, the individual mouse WDR79 and p53 promoters also show increasing activity in a dose dependent manner (FIG. 4B). These results demonstrate that this region of both the human and mouse genome is capable of driving gene expression in both orientations. It was concluded that the intergenic region between the TSS of p53 and the upstream flanking gene WDR79, therefore, is a bona fide bidirectional promoter.

Following identification of the bidirectionality of the p53 and WDR79 promoters, a way was developed to simultaneously study the transcriptional activities of both promoters in vitro. Specifically, mouse and human p53/WDR79 promoters were cloned into the pLucRLuc bidirectional expression vector at the Kpn1 cloning site. The DNA containing the mouse or human bidirectional p53/WDR79 promoters and a control β-Gal expressing construct were transiently transfected into murine Swiss 3T3 fibroblasts and human U2OS osteosarcoma cell lines, respectively. Increasing amounts of the bidirectional expression vector and a constant amount of the β-Gal expressing construct were co-transfected into the various cell lines and were allowed to incubate overnight. Extracts were obtained and Luciferase, Renilla, and β-Gal activity was assayed. Each titration was performed multiple times. All results were normalized to β-Gal activity.

Since the bidirectional expression vector was transcribing the p53 promoter in one direction, and the WDR79 promoter in the other, the activity of the two bioluminescent reporters was measured to determine the transcriptional output from each of the promoters. In all of these experiments, the WDR79 promoter was driving the Renilla reporter gene, while the p53 promoter was driving the transcription of the Luciferase gene. The results were normalized to the β-Gal activity (in which the DNA concentrations remained constant throughout all transfections).

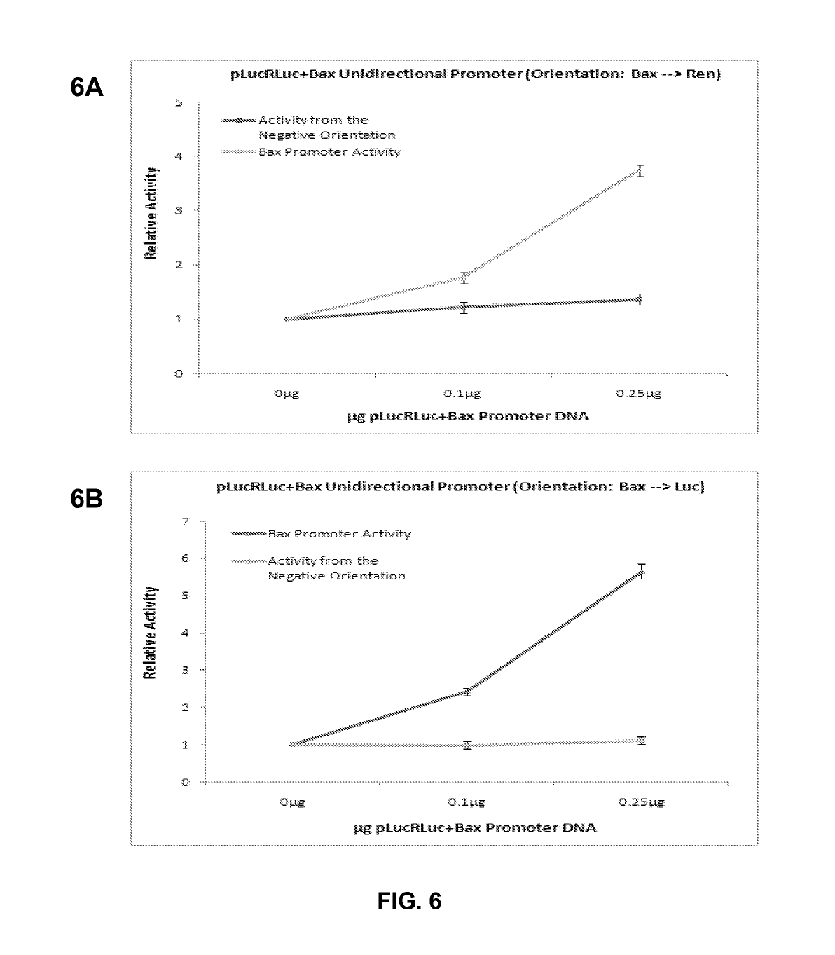

As seen in FIG. 5, both the murine (FIG. 5A) and human (FIG. 5B) WDR79 and p53 promoters showed an increase in transcriptional activity in a dose dependent manner. This is consistent with the findings assaying the individual promoter activities using the pGL3 expression vector (FIG. 4). Interestingly, in human U2OS cells, while the WDR79 promoter reached maximal expression at approximately 0.1 μg of DNA, the p53 promoter activity continues to increase as the amount of reporter DNA is increased, at least up to 0.5 μg (FIG. 5B). Once the transcription factors have been identified that regulate these promoters, it will be possible to determine whether this difference in activities is due, for example, to a factor that becomes limiting for WDR79 expression, but not for p53 expression. These results verify that the promoter sequences upstream of both the human and mouse p53 genes function bidirectionally and that the pLucRLuc bidirectional expression vector is a functional vector capable of transcribing bidirectional promoters.

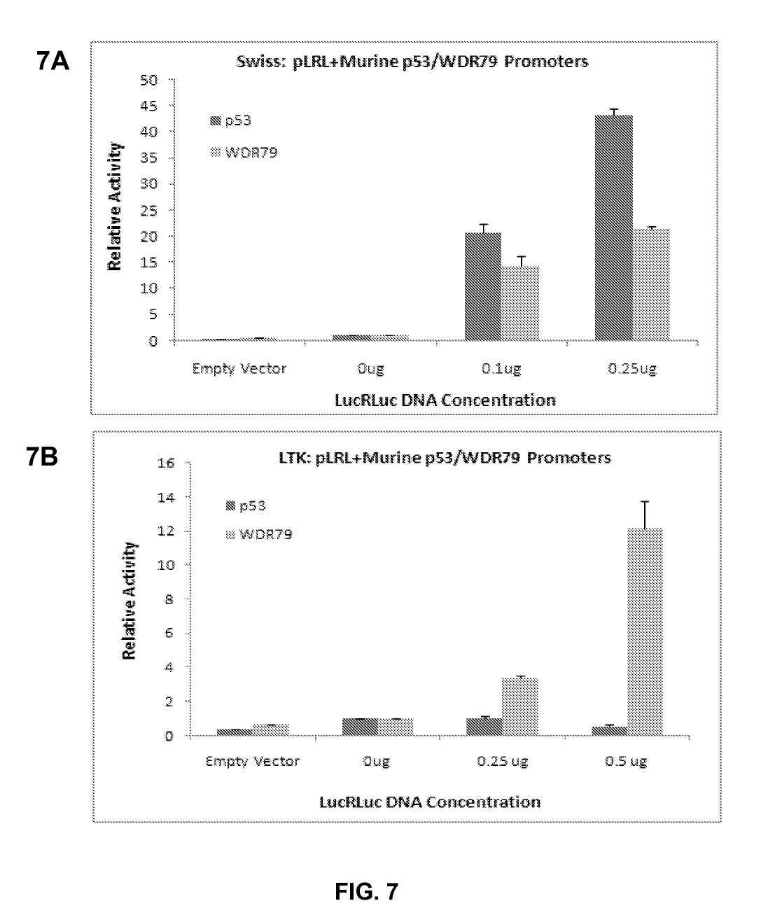

To establish that the reporter activity was due to the presence of a bidirectional promoter, the unidirectional human Bax promoter (Miyashita, T. and Reed J C, 1995) was also cloned into the pLucRLuc bidirectional expression vector in both orientations and transfected into murine Swiss3T3 cells. As seen in FIG. 6, the Bax promoter was the only sequence that was capable of driving its respective reporter gene. The activity of the negative orientation remained undetectable as increasing amounts of the pLucRLuc+Bax construct was transfected. The cloning of a unidirectional promoter into the bidirectional pLucRLuc construct led to the conclusion that the change in reporter activity was a direct result of the respective promoter that was driving its expression. Therefore, since no activity was seen in the negative orientation of the Bax sequence, it was concluded that the activity seen with the expression of the reporters driven by the p53 and WDR79 promoters was a result of these sequences being bidirectional promoters.

A representation of the activities obtained for each promoter can be seen in Table 1, below.

TABLE 1

| | Luciferase | Renilla |
|---|---|---|
| Vector/Insert | activity | Activity |
| pLucRLuc/empty | 0.35 | 0.67 |
| pLucRLuc/Mouse p53 → Ren | 22.75 | 2.04 |
| pLucRLuc/Mouse WDR79 → Ren | 43.06* | 21.33* |
| pLucRLuc/Human p53 → Ren | 21.87 | 106.16 |
| pLucRLuc/Human p53 → Ren | 181.4* | 161.34* |
| pLucRLuc/Bax → Luc | 5.65 | 1.11 |
| pLucRLuc/Bax → Ren | 1.36 | 3.74 |
| pLucRLuc/NF DNA | 0.96 | 1.28 |

In Table 1, the arrow after the gene name indicates the respective promoters driving their expression. Each number is the Relative Activity of the respective promoter and reporter gene when 0.25 μg of the corresponding construct was transfected into Swiss3T3 cells. Each value represents a fold change in activity as compared to 0 μg of the pLucRLuc+Insert (represented with a value of 1; not shown in table). The “Empty Vector” refers to the transfection of the pLucRLuc construct alone. The “NF DNA” represents a non-functional sequence of the p53 gene promoter. The “*” are indicative of the orientations that were used for transfections in subsequent experiments.

One observation that is not fully understood is that, while activity from the WDR79 and p53 promoters were consistently seen, the overall expression levels appear to vary as a function of the specific orientation in pLucRLuc. This can be seen in Table 1 when comparing, for example, pLucRLuc Mouse p53 Ren versus pLucRLuc Mouse WDR79→Ren. This may be due to the influence of specific DNA sequences located between the vector and the genomic insert that negatively influence transcription. This may become better understood as the proteins involved in the expression of these promoters are identified.

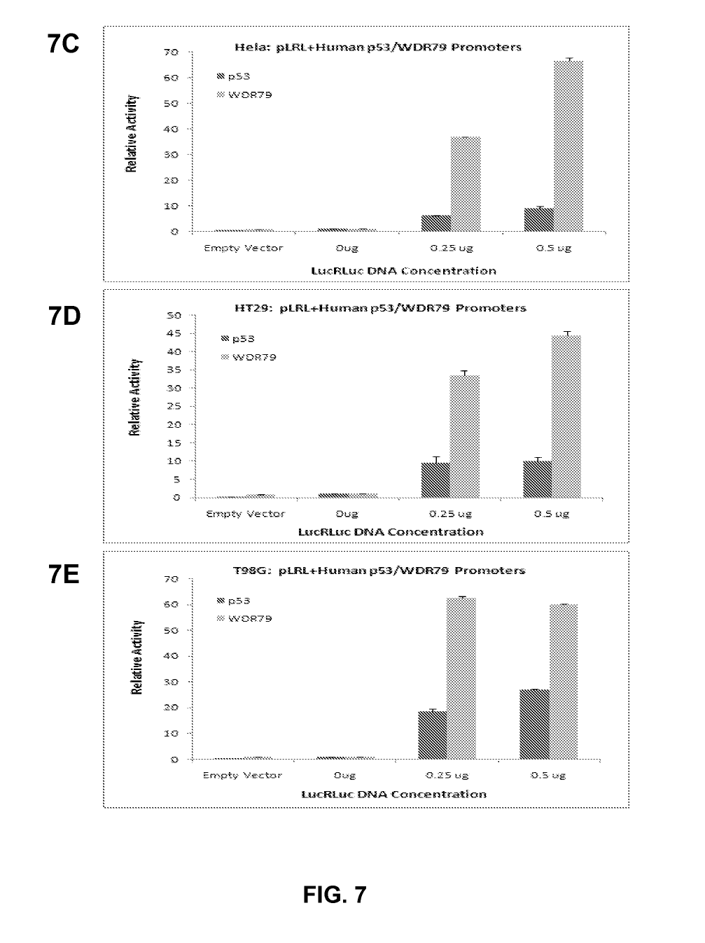

Thus, it was clearly demonstrated that the p53 gene and its bidirectional gene partner, WDR79, are expressed from the bidirectional expression vector (FIGS. 5 and 7). This was further validated in runs in which titrations of increasing increments of the pLucRLuc bidirectional expression vector containing the Bax promoter were transfected into Swiss3T3 cells. Each titration was performed in duplicate. Bax promoter activity and the activity of the sequence in the negative orientation were measured and the results were averaged. All results were normalized to β-Gal activity. Results are shown in FIG. 6, where it can be seen that the unidirectional human Bax promoter cloned into the pLucRLuc bidirectional expression vector only displayed activity when the wild type Bax promoter sequence was upstream of the respective reporter and that the activity of the negative orientation was zero.

In order to test the relative activities of the promoters, transient transfections were performed using various mouse and human cell lines. For these assays, the pLucRLuc construct containing the mouse p53/WDR79 bidirectional promoters were transiently transfected into the mouse non-transformed Swiss 3T3 and LTK fibroblastic cell lines and the pLucRLuc construct containing the human p53/WDR79 promoters were transiently transfected into the human HeLa, HT29, and T98G cancer cell lines.

Titrations of increasing increments of the pLucRLuc vector containing the mouse or the human bidirectional promoters and a constant concentration of the β-Gal construct was co-transfected into the various cell lines. All results were normalized to β-Gal activity and the relative activity of each promoter was obtained. As seen in FIG. 7, the activities of both promoters, as represented by reporter gene activity, increased as increasing amounts of the pLucRLuc bidirectional expression vector were introduced into cells.

In most cell lines, with the exception of Swiss 3T3, WDR79 promoter activity is higher relative to p53 activity (FIG. 7). In the LTK murine fibroblast cell line, although little or no activity of the p53 promoter was detected in LTK cells, the WDR79 promoter activity was quite high. These cells, therefore, could ultimately be quite useful for establishing the different requirements for activity of these two promoters. For example, a factor necessary for p53 expression, but not WDR79 expression, may be lacking in LTK cells. In fact, LTK cells express very low levels of endogenous, wild-type p53, consistent with the low level of promoter activity.

Likewise, in the human cells, WDR79 activity was consistently higher than p53. This could be related to the genetic makeup of each individual cell line or the overall function of the mouse and human promoters. HeLa, HT29, and T98G cells lines are cervical cancer, colon adenocarcinoma, and glioblastoma derived cell lines, respectively. However, the p53 status in each of these cell lines varies greatly. The HeLa cell line contains wild type p53, but is also infected with the human papilloma virus (HPV) (Zhao, et al, 2010). p53 is targeted for proteosomal degradation by the HPV E6 protein, therefore preventing the accumulation of functional p53 (Zhao, et al, 2010). The HT29 cell line is a p53 negative cell line (Ravizza, et al, 2004) while T98G is a mutant p53 cell line with non-functional wild type p53 (Ueda, et al, 1999). Additional experiments can be carried out to identify the factor(s) regulating these promoters and how this relates to their cell type specific expression.

Previous studies have shown that p53 is induced in early S-phase of the cell cycle by means of serum starvation and stimulation (Boggs and Reisman, 2006). Typically, the level of p53 mRNA is reduced in G₀ and is induced around 3-6 hours after entry into the cell cycle, reaching maximum levels by approximately 12-16 hours post-serum stimulation (Boggs and Reisman, 2006). In order to determine if both p53 and WDR79 are expressed simultaneously from the endogenous locus and whether there is any evidence of coordinated regulation, experiments were run to see if WDR79 also demonstrates this type of cell cycle regulation.

Swiss 3T3 cells were serum starved for 24 hrs in a reduced serum media (0.1% Fetal Bovine Serum) in order to force them into G₀. The cells were then stimulated to enter the cell cycle by adding an enriched media containing serum (15% FBS). The cells were harvested at various time points and their expression was monitored. RNA was extracted from exponentially growing (Exp), serum starved, or serum stimulated cells at various time points post-serum stimulation. The RNA was then converted to cDNA, and was subjected to PCR analysis. The PCR products were analyzed on a 1% agarose gel. Primer sets corresponding to p53, WDR79, and β-actin were used to amplify the respective mRNAs as described above.

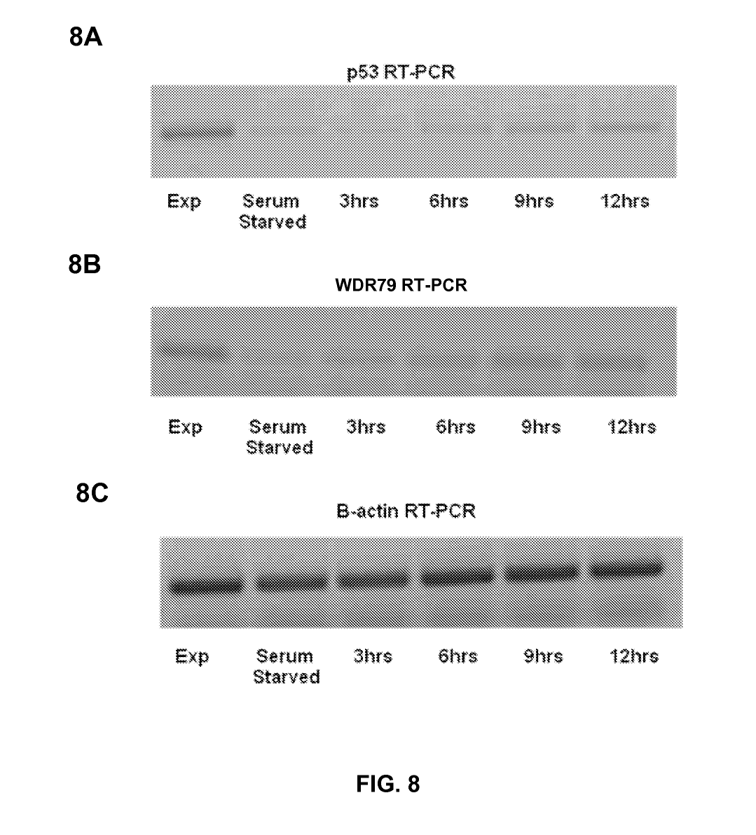

In accord with previous studies, the expression pattern of p53 was reduced after serum starvation for 24 hrs (FIG. 8A). As the cell cycle progressed, p53 expression increased (FIG. 8A). When analyzing the expression of WDR79, an analogous pattern was also seen (FIG. 8B). WDR79 levels were reduced in cells that have undergone serum starvation, while the expression was increased as the cells re-entered the cell cycle (FIG. 8B). β-actin expression was also analyzed as a control to show that there was no cyclical effect with this particular house-keeping gene (FIG. 8C). From this data, it was concluded that WDR79 was regulated in a cell cycle dependent manner, similar to that of p53 (compare FIG. 8A and FIG. 8B).

### REFERENCES

Alonso, S., et al. (2009). The diversity profile of TP53 is influenced by positive selection on the immediately upstream locus WDR79. Human Heredity, 69: 34-44.
Balint, E. and Reisman, D. (2006). Increased rate of transcription contributes to elevated expression of the mutant p53 gene in Burkitt's lymphoma cells. Cancer Res, 56(7):1648-53.
Boggs, K. and Reisman, D. (2006). Increased p53 transcription prior to DNA synthesis is regulated through a novel regulatory element within the p53 promoter. Oncogene 25, 555-565.
Boggs K, Reisman D. (2007). C/EBPβ participates in regulating transcription of the p53 gene in response to mitogen stimulation. The Journal of Biological Chemistry 2007; 282, 7982-7990.
Buratowski, S. (2008). Gene Expression—Where to Start? Science, Vol 322. pp 1804-1805.
De Wet, J., et al. (1986). Firefly Luciferase Gene: Structure and Expression in Mammalian Cells. Molecular and Cell Biology 7, 725-737.
Engstrom, P. G., et al. (2006). Complex loci of the human and mouse genome. PLoS Genet. 2(4), e47.
Fish, J., et al. (2007). Hypoxia-inducible expression of a cis-antisense transcript inhibits endothelial nitric-oxide synthase. JBC, 282; 15652-15666.
Garcia-Closas, M., et al. (2007). Common genetic variation in TP53 and its flanking genes, WDR79 and ATP1B2, and susceptibility to breast cancer. Int. J. Cancer: 121, 2532-38.
Khoo K, Mayer S, Fersht A R. (2009). Effects of Stability on the Biological Function of p53. Manuscript M109.033183.
Lapidot, M. and Pilpel, Y. (2006). Genome-wide natural antisense transcription: coupling its regulation to its different regulatory mechanisms. EMBO Reports, 7(12); 1216-22.
Lorenz, W. W., et al. (1991). Isolation and Expression of a cDNA encoding Renilla reniformis Luciferase. Proc Natl Acad Sci 88(10), 4438-4442.
Mahmoudi, S., et al. (2009). Wrap53, a natural p53 antisense transcript required for p53 induction upon DNA damage. Molecular Cell 33, 462-471.
Mahmoudi, S., et al. (2010). Wrap53 is essential for cajal body formation and for targeting the survival of motor neuron complex to cajal bodies. PLoS, 8(11), 1-16.
Misra, S., et al, (2010). Cell cycle-dependent regulation of the bi-directional overlapping promoter of human BRCA2/ZAR2 genes in breast cancer cells. Molecular Cancer, 9(50).
Miyashita, T. and Reed, J C. (1995). Tumor suppressor p53 is a direct transcriptional activator of the human Bax gene. Cell 80(2):293-9.
Pointkivska, H., et al. (2009). Cross-species mapping of bidirectional promoters enables prediction of unannotated 5′ UTRs and identification of species-specific transcripts. BMC Genomics, 10:189. doi:10.1186/1471-2164-10-189
Raman, V., et al. (2000). Compromised HOXA5 function can limit p53 expression in human breast tumours. Nature, 405(6789): 974-8.
Ravizzi, R., et al. (2004). Role of the p53/p21 system in the response of human colon carcinoma cells to doxorubicin. BMC Cancer, 4(92). doi:10.1186/1471-2407-4-92.
Reisman, D., et al. (1988). Human p53 oncogene contains one promoter upstream of exon 1 and a second, stronger promoter within exon 1. Proc. Natl. Acad. Sci. USA 85:5146-50.
Reisman, D., et al. (2001). Cloning and characterization of murine upstream sequences reveals
   positive regulatory elements. Gene, 274, 129-137.
Seila, A., et al. (2009). Divergent transcription. Cell Cycle, Vol 8; Issue 16, 2557-64.
Trinklein, N., et al. (2004). An abundance of bidirectional promoters in the human genome. Genome Research, 14:62-66.
Ueda, K., et al. (1999). Induction of apopotosis in T98G glioblastoma cells by transfection of GML, a p53 target gene. Oncol Res, 11; 125-32.
Ueda, R., et al. (2006). A common regulatory region functions bidirectionally in transcriptional activation of the human CYP1A1 and CYP1A2 genes. Molecular Pharmacology. 69, 6: 1924-30.
Vogelstein B, Lane D, Levine A J. (2000). Surfing the p53 network. Nature; 408, 307-310.
Vousden K H, Lu X. Live or let die: the cell's response to p53. (2002). Nature Reviews (Cancer) 2; 594-604.
Wang, Q., et al. (2009). Searching for bidirectional promoters in Arabidopsis thaliana. BMC Bioinformatics, 10(S29). doi: 10.1186/1471-2105-10-S1-S29.
Wright, K., et al. (1995). Coordinate regulation of the human TAP1 and LMP2 genes from a shared bidirectional promoter. J. Exp. Med. 181; 1459-71.
Yang, M., and Elnitski, L. (2008). Diversity of core promoter elements comprising human bidirectional promoters. BMC Genomics, 9(S3). doi: 10.1186/1471-2164-9-S2-S3
Yang, M., et al. (2008). Comparative analysis of bidirectional promoters in vertebrates. BMC Bioinformatics, 9(S9). doi: 10.1186/1471-2105-9-S6-S9
Zanotto, E., et al. (2006). The bidirectional promoter of two genes for the mitochondrial translational apparatus in mouse is regulated by an array of CCAAT boxes interacting with the transcription factor NF-Y. Nucleic Acids Research. 35, 2: 664-77.
Zhao Y, Chen X Q, Du J Z. Cellular adaptation to hypoxia and p53 transcription regulation. Journal of Zhejiang University Science. 2009 May; 10(5): 404-410.
Zhao, C Y, et al. (2010). Rescue of p53 function by small-molecule RITA in cervical cancer carcinoma by blocking E6 mediated degradation. Cancer Res, 70(8): 3372-81.

What is claimed is:

1. A bidirectional expression vector comprising a first reporter gene and a second reporter gene, the first and second reporter genes being arranged in a head to head (5′ to 5′) arrangement, the bidirectional expression vector including a polylinker region between the heads of the first and second reporter genes, the bidirectional expression vector including multiple cloning sites within the polylinker region.

2. The bidirectional expression vector of claim 1, wherein the first reporter gene is a renilla gene.

3. The bidirectional expression vector of claim 1, wherein the second reporter gene is a luciferase gene.

4. The bidirectional expression vector of claim 1, the polylinker region including more than one copy of at least one of the restriction sites.

6. The bidirectional expression vector of claim 1, the polylinker region including the following restriction sites: SmaI, XmaI, KpnI, SmaI, XmaI, and PstI, the restriction sites being inserted in the order as provided.

7. The bidirectional expression vector of claim 1, the polylinker region including at least one of each of SmaI, KpnI, SacI, SalI, XhoI, and BgII as restriction sites.

8. The bidirectional expression vector of claim 1, further including an antibiotic resistance gene.

9. The bidirectional expression vector of claim 8, wherein the antibiotic resistance gene is an ampicillin resistance gene.

10. The bidirectional expression vector of claim 1, further comprising a splicing signal.

11. The bidirectional expression vector of claim 1, further comprising a polyadenylation signal.

12. The bidirectional expression vector of claim 11, further comprising a first polyadenylation signal downstream of the first reporter gene and a second polyadenylation signal downstream of the second reporter gene.

13. A host cell comprising the bidirectional expression vector of claim 1.

14. The host cell of claim 13, wherein the host cell is a mammalian cell.

15. The host cell of claim 14, wherein the host cell is a human cell or a murine cell.

16. A kit comprising the bidirectional expression vector of claim 1.

17. A method for the production of a first expression product of a first reporter gene and a second expression product of a second reporter gene from a bidirectional vector, the method including:
cloning a polynucleotide into a cloning site of the bidirectional vector, the bidirectional vector including the first reporter gene and the second reporter gene in a head to head (5′ to 5′) arrangement and a polylinker region between the heads of the first and second reporter gene, the polylinker region including the cloning site and further including at least one additional cloning site;
transfecting a host cell with the bidirectional vector; and
culturing the host cell.

18. The method according to claim 17, further comprising determining the presence or quantity of at least one of the first and second expression products.

19. The method according to claim 17, further comprising incorporating a mutation into the polynucleotide.

20. The method according to claim 17, further comprising deleting a segment of the polynucleotide.
